(19) United States
(12) Patent Application Publication
(10) Pub. No.: US 2011/0280945 A1
(43) Pub. Date: Nov. 17, 2011

(54) NOVEL METHOD FOR THE PREPARATION OF GRANULATES OF ACTIVE CONSTITUENTS, AND GRANULATES AS OBTAINED

(75) Inventors: Christophe Lebon, Rouvres (FR); Pascal Suplie, Montaure (FR)

(73) Assignee: Debregeas ET Associes Pharma, Paris (FR)

(21) Appl. No.: 13/129,028

(22) PCT Filed: Nov. 13, 2009
(86) PCT No.: PCT/FR2009/052180

§ 371 Date: Jul. 26, 2011

(30) Foreign Application Priority Data
Nov. 14, 2008 (FR) 0857764

(51) Int. Cl. A61K 9/16; A61K 31/55; B05D 1/06; A61P 3/10; A61P 25/08; A61P 3/06; A61K 31/616; A61K 31/64

(52) U.S. Cl. 424/494; 424/490; 424/493; 424/497; 424/498; 424/496; 514/161; 514/217; 514/412; 427/2.14

(57) ABSTRACT

The present invention relates to a method for preparing a granulate of at least two active principles, including a step of applying said active principles to a solid particulate medium by dusting, said active principles not being plant extracts.

### CROSS-REFERENCE TO RELATED APPLICATIONS

This application is a national phase application of PCT/FR2009/052180 filed on Nov. 13, 2009 and claims priority to French Application No. 0857764 filed on Nov. 14, 2008. The above-identified patent applications are incorporated herein, by reference, in their entirety.

### FIELD OF THE INVENTION

The present invention relates to a novel method for the preparation of granulates of active constituents, as well as the granulates as obtained.

### BACKGROUND OF THE INVENTION

Numerous active constituents have pharmacokinetic profiles which involve a reduction in their efficacy, for example, in the case of a short half-life and/or a high plasma concentration peak and/or rapid elimination and/or low bioavailability.

Such pharmacokinetic profiles involve the administration of large daily doses and/or concomitant administrations repeated throughout the day, and also a limited efficacy owing to the great variations in plasma concentration and a risk of intolerance due to those same variations. In addition, this is prejudicial to the observance of the treatment.

There is therefore currently a need for the development of a galenic form bringing an improvement to that profile and reducing the number of administrations of medicaments, by enabling several active constituents to be combined within the same unit.

### SUMMARY OF THE INVENTION

An object of the present invention is to provide a method for the preparation of a novel galenic form enabling the above-mentioned disadvantages to be avoided.

Thus, an object of the present invention is to provide a novel galenic form enabling the daily dose and the number of daily administrations to be reduced, by increasing the apparent half-life and the bioavailability of the active constituents.

Thus, an object of the present invention is to provide a novel galenic form enabling the secondary effects to be reduced or suppressed by reducing the plasma concentrations used.

Thus, an object of the present invention is to provide a novel galenic form enabling the comfort of the patient and the monitoring of the treatment to be improved by reducing the number of daily administrations.

Thus, an object of the present invention is to provide a novel galenic form enabling the safety of the product to be improved by a stable galenic form.

### DETAILED DESCRIPTION OF THE INVENTION

The present invention relates to a method for the preparation of a granulate of at least two active constituents, comprising a step of applying the active constituents by powdering to a solid particulate support, characterised in that the active constituents are not plant extracts.

The expression “granulate” denotes a preparation composed of dry solid grains, each forming an aggregate of powder particles having sufficient solidity to allow various manipulations.

Generally, the granulates are in the form of small grains of substantially uniform size and of irregular angular shape. The granulates according to the present invention have the characteristic that they have a shape which is quite regular, quasi-spherical and fairly smooth.

From the physical point of view, the granulates are aggregates of various crystallised or amorphous powder particles.

The granulates of the present invention are intended for oral administration and they are more particularly intended to be swallowed just as they are.

The method of the invention therefore consists in mixing the active constituents in the form of a powder in the presence of solid particles as the support. Thus, the solid particles of the support used form a core on which the particles of the active constituents are deposited.

The implementation of the method of the invention thus enables granulates having a core-skin structure to be obtained.

By carrying out comparative tests for the preparation of granulates by a direct granulation method with various excipients customarily used in granulation, it has been found that the results obtained relating to the granulate itself are satisfactory with regard to appearance, friability and dissolution. However, the granulates obtained by such a method have a very large specific surface area requiring large quantities of polymers for coating in accordance with the techniques conventionally used.

Thus, the granulates of the present invention are characterised in that they have a smaller specific surface area. In addition, in appearance, they are relatively smooth and have a fairly regular shape.

Of the active constituents, mention may be made in particular of antipaludials, antibiotics, antihypertensives, antivirals (and antiretrovirals), antiepileptics, the active constituents used in gastroenterology, the active constituents used in dermatology, anticancer agents especially of the cisplatin type or 5-flurouracil, as well as hypolipaemics.

According to a particularly advantageous embodiment, the core of the granulates of the invention is not composed of particles having a sugar sphere. Preferably, the solid core of the granulates of the invention is not a sugar sphere.

The expression “sugar sphere” denotes a spherical solid support having a homogeneous surface state. In the context of the present invention, those supports are not advantageous because, on the one hand, they cause solubility problems (dissolution too slow) and, on the other hand, owing to their excessive regularity, they do not enable a homogeneous (granulated) final product to be obtained.

Owing to the large volume of granulates and for reasons of acceptance and therefore observance of the treatment, absorption must be rapid and easy and therefore be similar to a liquid form/ampoules. Various types of support have thus been tested.

The spherical supports tested, such as, for example, the sugar spheres of saccharose and starch, have not given satisfactory results in terms of final dissolution of the form.

Furthermore, their spherical surface is too regular, which represents an advantage in terms of coating but in the present case does not enable the small particles of adjuvants (flavourings, sweeteners) to cling and therefore is ultimately prejudicial to good homogeneity.

It is known to the person skilled in the art that it is very difficult to obtain a homogeneous mixture of powders because every powder has its own physico-chemical characteristics. In addition, the final form assumes the use of various adjuvants which themselves have different particle sizes.

In order to overcome that problem, it has been observed that, by mixing all of the various powders and by carrying out a crushing operation, a much more homogeneous mixture of defined particle size is obtained.

For the mixture can then “cling” in the anfractuosities of the support grain during the powdering operations in successive layers and therefore contribute to the rounding of the grain.

The various successive crushing and powdering operations are indispensable in obtaining the targeted particle size dispersion which makes it possible to respond simultaneously to the various constraints listed above.

The supports used in the context of the present invention, other than the sugar spheres, have the advantage of having a surface state which is not very homogeneous but which has anfractuosities in which the various active constituents will become fixed in the form of a powder. This choice is important in enabling a homogeneous final product to be obtained despite the mixture of at least two powders having different particle sizes.

Preferably, the solid core of the granulates of the invention is composed of particles having an average diameter of from 300 μm to 650 μm, preferably from 400 to 600 μm.

The granulated mannitol support, and more particularly the grade 400-500, is preferred because such a support has a sufficiently large size to enable smaller particles (less than 100 microns) to be fixed thereto.

It has thus been demonstrated that, by mixing the various active constituents with mannitol and by crushing the whole, a homogeneous mixture is obtained.

Thus, finally, a homogeneous granulate having a particle size centred around 500 microns is obtained.

And more especially, this granulate has the following particle size distribution: 20% of the particles have a diameter of less than 710 μm, 70% of the particles have a diameter of less than 500 μm and 25% of the particles have a diameter of less than 315 μm.

The above-mentioned powdering step of the method for the preparation of the granulates of the invention may also comprise a step of spraying an aqueous, alcoholic or hydroalcoholic solution of a binder.

Those spraying and powdering steps are preferably carried out simultaneously or alternately.

Preferably, the above-mentioned powdering step is carried out concomitantly with a step of spraying a binder in the form of a solution.

The combination of those steps provides for good cohesion of the active constituents on the core of the granulates.

An advantageous implementation of the method of the invention thus consists in applying the active constituents in the form of a powder to the above-mentioned particulate support (or core of the granulates) by alternating spraying sequences of the binder in the form of a solution.

As binders, mention may be made of the majority of the hydrophilic excipients which give viscous solutions: gum arabic and gum tragacanth, methylcellulose and carboxymethylcellulose, gelatin, starches, maltodextrins, PEG 4000 and 6000 in alcoholic solution, polyvidone in aqueous or alcoholic solution and also saccharose, glucose or sorbitol solutions.

According to a particular embodiment, the above-mentioned method also comprises, after the powdering step, a step of coating the granulate, in particular by depositing a coating agent in the form of a film on the granulate by lamination.

This coating step thus enables the granulates obtained to be consolidated and possibly ensures that the taste of the active constituents is masked.

The small specific surface area of the granulates of the invention thus permits, in the case of coating, a reduction in the amount of coating agent used and therefore a reduction in the dilution of the active constituents in the coated granulates.

A preferred embodiment of the method of the invention consists in a method comprising, after the coating step, a step of mixing with a lubricant and/or a flavouring and/or a sweetener and/or a colouring.

If necessary, the above-mentioned method may also comprise, before the powdering step, a step of crushing the active constituents in the presence of a diluent.

Thus, according to a preferred embodiment, the method for the preparation of the granulates of the invention comprises the following steps:
a step of applying the active constituents by powdering to a solid particulate support, combined with a step of spraying an aqueous, alcoholic or hydroalcoholic solution of a binder, in order to obtain a granulate, the granulate being composed of a core corresponding to the above-mentioned support on which particles of the active constituents are deposited;
one or more steps of coating the granulate obtained in the previous step, by depositing a coating film by lamination, in order to obtain a coated granulate; and
an optional step of mixing with a lubricant and/or a flavouring and/or a sweetener and/or a colouring.

A particularly advantageous method according to the present invention is a method in which the solid particulate support is selected from the group composed of polyols, such as mannitol, sorbitol, maltitol or xylitol, lactose, dicalcium phosphate, carbonates, such as calcium, potassium, magnesium or sodium carbonate, gluconates, silicates, sugar crystals, saccharose and silica derivatives.

Preferably, the solid particulate support does not comprise a cellulose compound. Preferably, the solid particulate support is not a sugar sphere.

According to a particularly preferred embodiment of the method of the invention, the solid particulate support is composed of mannitol. The granulates so obtained are composed of a core composed of mannitol particles around which the particles of active constituents are deposited.

Preferably, in the context of the implementation of the method of the invention, the binder is selected from the group composed of polyvinylpyrrolidone (PVP or polyvidone), hydroxypropylmethylcellulose (HPMC), shellac, hydroxypropylcellulose (HPC), cellulose, polyols, alginates, polyglycolysed glycerides (Gelucire®) or macrogolglycerides, especially stearoyl macrogolglycerides, as well as mixtures thereof.

Of the polyols, mention may be made in particular of mannitol, sorbitol, maltitol or xylitol.

According to a particular embodiment, the binders used in the method according to the present invention are not cellulose compounds. They are therefore preferably selected from the group composed of polyvinylpyrrolidone, shellac, polyols and alginates, polyglycolysed glycerides or macrogolglycerides, especially stearoyl macrogolglycerides, as well as mixtures thereof.

Of the coating agents used in the context of the method of the invention, it is preferable to use coating agents selected from the group composed of shellac, polyvinylpyrrolidone, polyethylene glycol, cellulose derivatives, such as HPMC or HPC, saccharose, alginate, methacrylic polymers and glycerides of fatty acids, or any other pharmaceutically acceptable coating polymer.

The present invention relates also to a method for the preparation of a granulate comprising an enteric coating, the method comprising a step of applying a coating agent composed of HPMCP (hydroxypropylmethylcellulose phthalate-hypromellose phthalate) or methacrylic polymers, especially Eudragit® L30D, or shellac.

The presence of this enteric coating may enable the bioavailability of the active constituents to be increased, avoiding their degradation in an acidic environment.

The present invention relates also to a method for the preparation of a granulate comprising a coating for prolonged release, the method comprising one or more steps of applying a coating agent composed of copolymers of methacrylates and acrylates Eudragit® RL, Eudragit® L100, shellac, derivatives of cellulose, especially ethylcellulose, and acrylic derivatives.

The granulates so obtained permit modified or delayed release of the active constituents (modified release granulates).

The presence of this coating for modified release makes it possible, in particular, to increase the apparent half-life of the active constituents.

The present invention relates also to a granulate which can be obtained in accordance with the method as defined above.

The present invention relates also to a granulate of at least two active constituents, characterised in that it comprises a solid core on which the active constituents are supported and in that the active constituents are not plant extracts.

The granulates of the present invention have a characteristic structure of the core-skin type, the core not being of the same nature as the active constituents forming the skin.

Thus, these granulates have a multi-layer structure. The active constituents are deposited on the core and therefore form a layer (or skin) deposited around that core (or support).

The core of the granulates may also be regarded as being a support on which the particles of the active constituents will become fixed.

The core is composed of solid particles and the active constituents supported by the core are also in a solid form.

The present invention is therefore based on the development of a novel multi-particle oral form.

Thus, the original nature of the form presented here consists in a granulate for oral administration, permitting the administration of at least two active constituents other than plant extracts at sufficiently high doses to require only one or two administrations per day, the granulate of the invention being highly concentrated in active constituents.

The granulates of the present invention have the advantage of reducing the number of daily administrations. Thus, given that the granulates of the invention are highly dosed, the amount of active constituents per dose unit (that is to say, per individual container containing the granulates, in particular a plastics ampoule) is preferably greater than or equal to 500 mg, advantageously greater than or equal to 1 g, and preferably greater than or equal to 1.5 g.

The granulates of the present invention have the advantage of permitting a reduction, for the patient, of the number of daily administrations.

According to a preferred embodiment, the core of the granulates of the invention is composed of particles of a compound selected from the group composed of polyols, such as mannitol, sorbitol, maltitol or xylitol, lactose, dicalcium phosphate, carbonates, such as calcium, potassium, magnesium or sodium carbonate, gluconates, silicates, in particular magnesium aminosilicate (Neusilin®) sugar crystals or saccharose.

According to a particularly preferred embodiment, the core of the granulates of the invention is composed of mannitol.

Preferably, the present invention therefore relates to granulates comprising particles of active constituents deposited on a core composed of mannitol particles.

The granulates according to the present invention may also comprise a binder.

The role of the binder is to bind the particles to each other, that is to say, to perfect the cohesion of the granulate. Thus, the binders provide for a good cohesion of the active constituents and the core in the granulates and for the rounding of the granulate.

Thus, the binders, like the active constituents, are deposited around the core of the granulates.

The binders of the granulates of the invention are preferably selected from the group composed of starch, saccharose, gum arabic, polyvinylpyrrolidone (PVP or polyvidone), hydroxypropylmethylcellulose (HPMC), shellac, hydroxypropylcellulose (HPC), cellulose, polyols or alginates, polyglycolysed glycerides (Gelucire®) or macrogolglycerides, especially stearoyl macrogolglycerides, as well as mixtures thereof.

According to a particular embodiment, the binders used in the granulates of the present invention are not cellulose compounds.

According to a particular embodiment, the granulates of the invention are coated.

The coated granulates are composed of grains coated with one or more layers of mixtures of various excipients.

Thus, the preferred coated granulates according to the present invention comprise the active constituents deposited on a core composed of mannitol particles, as well as an additional layer composed of coating agent(s).

According to a preferred embodiment, the granulates of the invention have a multi-layer structure and are composed of a core, preferably based on mannitol, on which are deposited the active constituents and the binder, which are themselves coated with one or more layers of coating agent(s).

The granulates of the invention are preferably coated with one or more coating agents selected from the group composed of shellac, polyvinylpyrrolidone, polyethylene glycol (PEG), cellulose derivatives, such as HPMC or HPC, saccharose, alginate and glycerides of fatty acids.

According to a particularly preferred embodiment, the granulates of the invention are coated with shellac.

The granulates of the invention may also be coated with one or more coating films to which one or more excipients, such as lubricants, colourings or sweeteners, are added.

The granulates of the invention may also contain one or more plasticisers, such as those conventionally used by the person skilled in the art.

The granulates of the invention may also comprise an enteric coating for gastric protection. Such granulates are therefore gastro-resistant.

Such a coating is obtained with coating agents composed in particular of HPMCP (hydroxypropylmethylcellulose phthalate-hypromellose phthalate) or methacrylic polymers, in particular Eudragit® L30D, or shellac.

The granulates of the invention may also comprise a coating for prolonged release.

Such granulates permit modified or delayed release of the active constituents (modified release granulates).

Such a coating is obtained with coating agents which are composed, in particular, of copolymers of methacrylates and acrylates Eudragit®RL, Eudragit® L100, shellac, derivatives of cellulose, especially ethylcellulose, and acrylic derivatives.

The granulates according to the present invention may also comprise a lubricant and/or a flavouring and/or a sweetener and/or a colouring.

The lubricants, flavourings, sweeteners and colourings which may be present in the granulates of the invention are especially as defined above.

Particularly preferably, the granulates according to the present invention are characterised in that the core represents from 10 to 70%, and preferably from 25 to 55% by weight relative to the total weight of the granulate.

Preferably, a granulate according to the present invention comprises at least 20% by weight of active constituents, and especially from approximately 30% to approximately 60% by weight.

The granulates of the invention preferably comprise less than 2% by weight of flavouring.

The granulates of the invention preferably comprise less than 1.5% by weight of colouring.

The granulates of the invention preferably comprise less than 2% by weight of sweetener.

The granulates of the invention preferably comprise less than 4% by weight of lubricant.

### EXAMPLES

### Detailed Description of a Preferred Embodiment for the Preparation of Granulates

The constituents are weighed one by one, then the active constituents are introduced into a cubic mixer (of the CMS type). The quantity of diluent is weighed in its turn (mannitol 160) and introduced into the mixer. The mixer is then set in operation. The mixture obtained (A) is satisfactory after 10 minutes.

The mixture is then introduced into a Forplex FLO mill and all of the mixture is crushed in such a way as to reduce the particle size of the whole (active constituents+diluent). This makes it possible to increase the difference in size of the particles of mannitol (support) (approximately 300μ) and of the crushed mixture (less than 100μ and preferably 25μ).

The following step of the method is a step of powdering in which the equipment used is a conventional turbine.

Thus the mannitol which serves as support is introduced into a vessel, this latter is then set in rotation (approximately 20 rotations per minute) and the mixture A is deposited by sequential powdering on the mannitol support, alternating with phases of spraying of the binder solution (PVP/HPMC/OH/H₂O).

This step is carried out sequentially in order to enable the evaporation and the drying of the granulates.

At the end of the step of powdering, a drying phase is carried out in order to cause hot air at approximately 40° C. to circulate over the mass of granulates for approximately 14 hours.

At the end of the drying step, the product is sieved in such a way as to select the particles obtained. The mixture is then returned to the vessel.

The following step is the step of coating. The solutions (or suspensions) containing the coating agents are placed successively in a low-pressure vessel subjected to agitation. The mass of granulates obtained is then placed in the vessel of a fluidised-air bed and the coating solutions are then sprayed successively in a continuous manner onto the granulates. Steps of drying/coating may also be carried out.

An apparatus of the fluidised-air bed type (or similar technology) is preferably used for the step of coating due to its great effectiveness in terms of evaporation, which makes it possible to considerably reduce the coating times.

Different types of coating may also be produced which each play a particular role, namely: consolidation, production of a hydrophobic layer, colouring, bitterisation, modification of the release of the active constituents.

Afterwards, the additives such as sweeteners, lubricants, flavourings and colourings may be added to the granulates in a mixer.

The last step consists in distributing the granulates into individual packages such as plastic ampoules or sachets.

The following tables describe examples of granulates obtained within the context of the present invention.

### Combination of Gliclazide/Metformin

### (Active Constituents Used in the Context of Treatment of Diabetes)

FORMULA No. 1

| | mg | % |
|---|---|---|
| Gliclazide | 60.00 | 2.61 |
| Metformin | 850.00 | 36.96 |
| Mannitol (support) | 714.75 | 31.08 |
| Calcium carbonate (support) | 225.00 | 9.78 |
| PVP/GLDB | 170.25 | 7.40 |
| GLDB (shellac) | 140.00 | 6.09 |
| Talc | 140.00 | 6.09 |
| Theoretical mass | 2300.00 | 100.00 |
| Theoretical content | 395.65 | |

FORMULA No. 2

| | mg | % |
|---|---|---|
| Gliclazide | 60.00 | 2.61 |
| Metformin | 850.00 | 36.96 |
| Mannitol (support) | 973.50 | 42.33 |
| PVP/GLDB | 136.50 | 5.93 |
| GLDB | 140.00 | 6.09 |
| Talc | 140.00 | 6.09 |
| Theoretical mass | 2300.00 | 100.00 |
| Theoretical content | 395.65 | |

FORMULA No. 3

| | mg | % |
|---|---|---|
| Gliclazide | 60.00 | 2.61 |
| Metformin | 850.00 | 36.96 |
| Neutrals 425-500 (support) | 714.75 | 31.08 |
| Calcium carbonate (support) | 225.00 | 9.78 |
| PVP/GLDB | 170.25 | 7.40 |
| GLDB | 140.00 | 6.09 |
| Talc | 140.00 | 6.09 |
| Theoretical mass | 2300.00 | 100.00 |
| Theoretical content | 395.65 | |

FORMULA No. 4

| | mg | % |
|---|---|---|
| Gliclazide | 60.00 | 2.61 |
| Metformin | 850.00 | 36.96 |
| Neutrals 425-500 (support) | 973.50 | 42.33 |
| PVP/GLDB | 136.50 | 5.93 |
| GLDB | 140.00 | 6.09 |
| Talc | 140.00 | 6.09 |
| Theoretical mass | 2300.00 | 100.00 |
| Theoretical content | 395.65 | |

### Combination of Carbamazipine/Sodium Valproate

### (Active Constituents Used as Antiepileptics)

FORMULA No. 5

| | mg | % |
|---|---|---|
| Carbamazipine | 400.00 | 26.67 |
| Sodium valproate | 200.00 | 13.33 |
| Mannitol (support) | 336.51 | 22.43 |
| Calcium carbonate (support) | 168.25 | 11.22 |
| PVP/GLDB | 115.24 | 7.68 |
| GLDB | 140.00 | 9.33 |
| Talc | 140.00 | 9.33 |
| Theoretical mass | 1500.00 | 100.00 |
| Theoretical content | 400.00 | |

FORMULA No. 6

| | mg | % |
|---|---|---|
| Carbamazipine | 400.00 | 26.67 |
| Sodium valproate | 200.00 | 13.33 |
| Mannitol (support) | 530.00 | 35.33 |
| PVP/GLDB | 90.00 | 6.00 |
| GLDB | 140.00 | 9.33 |
| Talc | 140.00 | 9.33 |
| Theoretical mass | 1500.00 | 100.00 |
| Theoretical content | 400.00 | |

FORMULA No. 7

| | mg | % |
|---|---|---|
| Carbamazipine | 400.00 | 26.67 |
| Sodium valproate | 200.00 | 13.33 |
| Neutrals 425-500 (support) | 336.51 | 22.43 |
| Calcium carbonate (support) | 168.25 | 11.22 |
| PVP/GLDB | 115.24 | 7.68 |
| GLDB | 140.00 | 9.33 |
| Talc | 140.00 | 9.33 |
| Theoretical mass | 1500.00 | 100.00 |
| Theoretical content | 400.00 | |

FORMULA No. 8

| | mg | % |
|---|---|---|
| Carbamazipine | 400.00 | 26.67 |
| Sodium valproate | 200.00 | 13.33 |
| Neutrals 425-500 (support) | 530.00 | 35.33 |
| PVP/GLDB | 90.00 | 6.00 |
| GLDB | 140.00 | 9.33 |
| Talc | 140.00 | 9.33 |
| Theoretical mass | 1500.00 | 100.00 |
| Theoretical content | 400.00 | |

### Combination of Simvastatin/Aspirin

### (For Hypercholesterolemia)

FORMULA No. 9

| | mg | % |
|---|---|---|
| Simvastatin | 40.00 | 4.44 |
| Aspirin | 160.00 | 17.78 |
| Mannitol (support) | 403.11 | 44.79 |
| Calcium carbonate/citric acid and/or | 201.56 | 22.39 |
| ascorbic acid (support) | | |
| PVP/GLDB | 60.23 | 6.69 |
| HPMC | 17.55 | 1.95 |
| Talc | 17.55 | 1.95 |
| Theoretical mass | 900.00 | 100.00 |
| Theoretical content | 222.22 | |

FORMULA No. 10

| | mg | % |
|---|---|---|
| Simvastatin | 40.00 | 4.44 |
| Aspirin | 160.00 | 17.78 |
| Mannitol (support) | 634.89 | 70.54 |
| Calcium carbonate/citric acid and/or | | 0.00 |
| ascorbic acid (support) | | |
| PVP/GLDB | 30.00 | 3.33 |
| HPMC | 17.55 | 1.95 |
| Talc | 17.55 | 1.95 |
| Theoretical mass | 900.00 | 100.00 |
| Theoretical content | 222.22 | |

FORMULA No. 11

| | mg | % |
|---|---|---|
| Simvastatin | 40.00 | 4.44 |
| Aspirin | 160.00 | 17.78 |
| Neutrals 425-500 (support) | 403.11 | 44.79 |
| Calcium carbonate/citric acid and/or | 201.56 | 22.39 |
| ascorbic acid (support) | | |
| PVP/GLDB | 60.23 | 6.69 |
| HPMC | 17.55 | 1.95 |
| Talc | 17.55 | 1.95 |
| Theoretical mass | 900.00 | 100.00 |
| Theoretical content | 222.22 | |

FORMULA No. 12

| | mg | % |
|---|---|---|
| Simvastatin | 40.00 | 4.44 |
| Aspirin | 160.00 | 17.78 |
| Neutrals 425-500 (support) | 634.89 | 70.54 |
| Calcium carbonate/citric acid and/or | | 0.00 |
| ascorbic acid (support) | | |
| PVP/GLDB | 30.00 | 3.33 |
| HPMC | 17.55 | 1.95 |
| Talc | 17.55 | 1.95 |
| Theoretical mass | 900.00 | 100.00 |
| Theoretical content | 222.22 | |

### Combination of Clopidogrel/Aspirin

FORMULA No. 13

| | mg | % |
|---|---|---|
| Clopidogrel hydrogen sulphate | 75.00 | 7.50 |
| Aspirin | 160.00 | 16.00 |
| Mannitol (support) | 441.05 | 44.10 |
| Calcium carbonate | 220.52 | 22.05 |
| (support) | | |
| PVP/GLDB | 68.33 | 6.83 |
| HPMC | 17.55 | 1.76 |
| Talc | 17.55 | 1.76 |
| Theoretical mass | 1000.00 | 100.00 |
| Theoretical content | 235.00 | |

FORMULA No. 14

| | mg | % |
|---|---|---|
| Clopidogrel hydrogen sulphate | 75.00 | 7.50 |
| Aspirin | 160.00 | 16.00 |
| Mannitol (support) | 694.64 | 69.46 |
| Calcium carbonate | | 0.00 |
| (support) | | |
| PVP/GLDB | 35.25 | 3.53 |
| HPMC | 17.55 | 1.76 |
| Talc | 17.55 | 1.76 |
| Theoretical mass | 1000.00 | 100.00 |
| Theoretical content | 235.00 | |

FORMULA No. 15

| | mg | % |
|---|---|---|
| Clopidogrel hydrogen sulphate | 75.00 | 7.50 |
| Aspirin | 160.00 | 16.00 |
| Neutrals 425-500 (support) | 441.05 | 44.10 |
| Calcium carbonate (support) | 220.52 | 22.05 |
| PVP/GLDB | 68.33 | 6.83 |
| HPMC | 17.55 | 1.76 |
| Talc | 17.55 | 1.76 |
| Theoretical mass | 1000.00 | 100.00 |
| Theoretical content | 235.00 | |

FORMULA No. 16

| | mg | % |
|---|---|---|
| Clopidogrel hydrogen sulphate | 75.00 | 7.50 |
| Aspirin | 160.00 | 16.00 |
| Neutrals 425-500 (support) | 694.64 | 69.46 |
| Calcium carbonate (support) | | 0.00 |
| PVP/GLDB | 35.25 | 3.53 |
| HPMC | 17.55 | 1.76 |
| Talc | 17.55 | 1.76 |
| Theoretical mass | 1000.00 | 100.00 |
| Theoretical content | 235.00 | |

1. A method for the preparation of a granulate of at least two active constituents, comprising a step of applying the active constituents by powdering to a solid particulate support, the active constituents not being plant extracts.

2. The method of claim 1, wherein the powdering step comprises spraying an aqueous, alcoholic or hydroalcoholic solution of a binder.

3. The method of claim 1, comprising, after the powdering step, a step of coating the granulate, followed if appropriate by a step of mixing with at least one of a lubricant, a flavouring, a sweetener, or a colouring.

4. The method of claim 1, wherein the support is selected from the group composed of polyols, lactose, dicalcium phosphate, carbonates, gluconates, silicates, sugar crystals, saccharose and silica derivatives.

5. The method of claim 2, wherein the binder is selected from the group composed of starch, saccharose, gum arabic, polyvinylpyrrolidone, hydroxypropylmethylcellulose, shellac, hydroxypropylcellulose, cellulose, polyols, alginates, polyglycolysed glycerides and macrogolglycerides.

6. A granulate which can be obtained according to the method according to claim 1.

7. A granulate of at least two active constituents, the active constituents not being plant extracts, comprising a solid core, on which the active constituents are supported.

8. The granulate of claim 7, wherein the solid core is not a neutral core.

9. The granulate of claim 8, comprising a binder.

10. The granulate of claim 8, wherein it is coated.

11. The granulate of claim 8, wherein the core represents from 10% to 70%, by weight relative to the total weight of the granulate.

12. The method of claim 3, wherein the step of coating the granulate consists in depositing a coating agent in the form of a film on the granulate by lamination.

13. The method of claim 4, wherein the polyols are chosen from the group consisting of mannitol, sorbitol, maltitol and xylitol.

14. The method of claim 4, wherein the carbonates are chosen from the group consisting of calcium carbonate, potassium carbonate, magnesium carbonate or sodium carbonate.

15. The granulate of claim 7, wherein the core is selected from the group consisting of polyols, lactose, dicalcium phosphate, carbonates, gluconates, silicates, sugar crystals, saccharose and silica derivatives.

16. The granulate of claim 15, wherein the polyols are chosen from the group consisting of mannitol, sorbitol, maltitol and xylitol.

17. The granulate of claim 15, wherein the carbonates are chosen from the group consisting of calcium carbonate, potassium carbonate, magnesium carbonate or sodium carbonate.

18. The granulate of claim 9, wherein the binder is selected from the group consisting of starch, saccharose, gum arabic, polyvinylpyrrolidone, hydroxypropylmethylcellulose, shellac, hydroxypropylcellulose, cellulose, polyols, alginates, polyglycolysed glycerides and macrogolglycerides.

19. The granulate of claim 10, wherein the granulate is coated by a coating agent chosen from the group consisting of shellac, polyvinylpyrrolidone, polyethylene glycol, cellulose derivatives, saccharose, alginate, glycerides of fatty acids and methacrylic polymers.
